(19) United States
(12) Patent Application Publication
(10) Pub. No.: US 2025/0170393 A1
(43) Pub. Date: May 29, 2025

(54) ADAPTIVE ELECTROPORATION

(71) Applicant: Cochlear Limited, Macquarie University, NSW (AU)

(72) Inventor: Paul Michael CARTER, Galson, NSW (AU)

(21) Appl. No.: 18/839,162

(22) PCT Filed: Feb. 21, 2023
(86) PCT No.: PCT/IB2023/051575

§ 371 Date: Aug. 16, 2024

Related U.S. Application Data
(60) Provisional application No. 63/325,885, filed on Mar. 31, 2022.
(60) Provisional application No. 63/312,904, filed on Feb. 23, 2022.

(51) Int. Cl. A61N 1/32; A61N 1/02; A61N 1/05; A61N 1/08; G16H 20/40; G16H 40/67

(52) U.S. Cl. CPC A61N 1/327; A61N 1/025; A61N 1/0541; G16H 20/40; G16H 40/67; A61N 2001/083

(57) ABSTRACT

Presented herein are adaptive electroporation techniques in which the electroporation parameters are adjusted/adapted based on the location within a body chamber, such as the cochlea, at which the electroporation electrical field is generated.

### CROSS-REFERENCE TO RELATED APPLICATIONS

This application also claims the benefit of the following U.S. Provisional Application No. 63/312,904, entitled “Adjustment of Electroporation Parameters,” filed Feb. 23, 2022, and U.S. Provisional Application No. 63/325,885, entitled “Adaptive Electroporation,” Mar. 31, 2022. These applications are hereby incorporated by reference herein.

### BACKGROUND

### Field of the Invention

The present invention relates generally to adaptive electroporation of cells based on the anatomical features of the area being electroporated.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: positioning an electroporation stimulation assembly including at least one electroporation electrode contact into a body chamber having a variable cross-sectional dimension; generating, via the at least one electroporation electrode contact, a first electroporation electrical field at a first location of the body chamber; and controlling one or more parameters used to generate the first electroporation electrical field to account for the variable cross-sectional dimension of the body chamber at the first location.

In another aspect, a method is provided. The method comprises: obtaining an impedance measurement from an intracavitary electrode assembly; and determining a voltage or current for electroporation of cells within a cavity from the impedance measurement.

In another aspect, a method is provided. The method comprises: inserting an electrode assembly comprising at least one electrode contact into an elongate body chamber having a variable cross-sectional dimension; and generating a substantially uniform electroporation electrical field along a length of the elongate body chamber as the electrode assembly moves through the variable cross-sectional dimension.

In another aspect, one or more non-transitory computer readable storage media are provided. The one or more transitory computer readable storage media comprise instructions that, when executed by a processor, cause the processor to: receive intracavitary electrical measurements from an electrode array; and determine one or more parameters for an electroporation electrical field from the intracavitary electrical measurements; wherein the one or more parameters control a strength of the electroporation electrical field.

In another aspect, a system is provided. The system comprises: an intracavitary electrode array configured to be inserted into a cavity within a body of a recipient: a measurement circuit configured to capture one or more electrical measurements from the intracavitary electrode array; and one or more processors configured to: determine least one of voltage or current for an electroporation electrical field from the electrical measurements, and control the at least one of voltage or current to set a strength of the electroporation electrical field.

### BRIEF DESCRIPTION OF THE DRAWINGS

Embodiments of the present invention are described herein in conjunction with the accompanying drawings, in which:

FIG. 1 is a schematic diagram illustrating an electroporation stimulation assembly inserted in a cochlea, in accordance with certain embodiments presented herein;

FIG. 2 is a schematic diagram illustrating a stand-alone electroporation sub-system, in accordance with certain embodiments presented herein;

FIG. 3A is a schematic diagram illustrating a cochlear implant system comprising an electroporation sub-system, in accordance with certain embodiments presented herein;

FIG. 3B is another schematic diagram illustrating the cochlear implant system of FIG. 3A, in accordance with certain embodiments presented herein;

FIG. 4 is a schematic diagram illustrating another cochlear implant system, in accordance with certain embodiments presented herein;

FIG. 5 is a flowchart of a method, in accordance with certain embodiments presented herein;

FIG. 6 is a flowchart of another method, in accordance with certain embodiments presented herein;

FIG. 7 is a flowchart of still another method, in accordance with certain embodiments presented herein; and

FIG. 8 is a schematic diagram illustrating a vestibular stimulator system with which aspects of the techniques presented herein can be implemented.

### DETAILED DESCRIPTION

Electroporation refers to the application of an electrical field to a cell in a manner that creates an electric potential (i.e., voltage difference) across the cell membrane that, in turn, temporarily opens up pores in the lipid bilayer membrane of the cell. The electrically opened pores can be used to, for example, allow a treatment material to enter the interior of the cell through the cell membrane (i.e., as the potential difference is applied to the cell, the electrically opened pores in the cell membrane allow material to flow into the cell). In certain examples, electroporation is used to open the pores in the cells in the presence of treatment materials including/comprising a therapeutic agent in order to enable an effective amount of the therapeutic agent to enter the electroporated (opened) cells. As used herein, the term “therapeutic agent” can include, but is not limited to, biological or bioactive substances, chemicals, pharmaceutical agents, nanoparticles, ions, genetic material including nucleic acids (e.g., DNA, DNA cassettes, cDNA, or plasmids, Ribonucleic acid (RNA) molecules, RNAi, etc.), proteins, peptides (e.g., Brain-derived neurotrophic factors, etc.), hormones, etc. After the electrical potential is removed, the pores in the cell membrane close such that the treatment material remains in the cell. As such, electroporation can be useful with medical implants by altering the biological composition of the cells in a manner that enhances, enables, etc. operation of the medical implants. It is noted that, with larger molecules such as DNA, the entire molecule may not enter the cell at the time of electroporation. Rather, the electroporation field drives the electrically charged molecule onto the surface of the cell where it sticks, to be taken up by the cell in the few seconds or minutes following electroporation.

For successful electroporation, a cell is typically exposed to an electrical field for a sufficient amount of time that enables a desired treatment material to migrate through the cell membrane. Such an electrical field, sometimes referred to herein as an “electroporation electrical field,” has a local field strength in the range of approximately 50 micro Volts per micro meter (uV/um) to approximately 500 uV/um. In general, the voltage needed to create a field having a strength in this range that is sufficient for electroporation heavily depends on the geometry of the anatomical area (e.g., body chamber) at which the electroporation electrical field is generated, the relative position of the electrode(s) to anatomical structures (e.g., walls of the body chamber), the distance between the anode and cathode electrode contacts used to generate the electroporation electrical field, and the characteristics of the medium(s) between electrode contacts and cells to be electroporated.

Electroporation can be delivered via an electroporation stimulation assembly including one or more electroporation electrode contacts (electroporation electrodes) that are disposed in (e.g., positioned in/on) a carrier member. Electroporation can be delivered via a stand-alone electroporation sub-system (e.g., an electroporation sub-system that is separate from an implantable medical device) or via an electroporation sub-system that is partially or fully integrated with an implantable medical device. For example, in certain embodiments, the electroporation electrode contacts are electrically connected to an external current or voltage source (e.g., a current or voltage source located outside of the body of the recipient). In other embodiments, the electroporation electrode contacts are electrically connected to an implantable current or voltage source (e.g., a current or voltage source located within the body of the recipient). In either case, the term “electroporation device” is sometimes used herein to refer to the current or voltage source that generates the electroporation signals. In addition, the term “electroporation sub-system” is sometimes used herein to refer to the combination of the electroporation electrode contacts and the current or voltage source.

Merely for ease of illustration, the embodiments presented herein are primarily described with reference to electroporation of a specific area of a body, namely the cochlea of a recipient, and with reference to a specific implantable medical device system, namely a cochlear implant system. However, it is to be appreciated that the techniques presented herein can also be used to electroporate a number of different areas in the body of a recipient and/or can be used with a number of different types of implantable medical device systems.

For example, the techniques presented herein can be implemented by, or used in conjunction with, other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by, or used in conjunction with, dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, dedicated electroporation systems, etc.

In intracochlear electroporation, a therapeutic agent is introduced into cells (e.g., nerve cells) within the cochlea for therapeutic benefit. In this process, an appropriate therapeutic agent is first introduced into the cochlea (e.g., suspended in a suitable conducting fluid) and an electroporation stimulation assembly is introduced into the cochlea. With the electroporation stimulation assembly in place, a predetermined set of electroporation signals (e.g., current pulses) are briefly applied through contacts on the array to generate an electrical field at the cells of the cochlea and thereby introduce the therapeutic agent into the cells. The pulses are typically applied sequentially at more than one location along the length of the array.

Electroporation is most effective and safe when the electrical field applied at the target cells to be electroporated is within an optimal range. An electrical field that is too low does not electroporate the cells, and therefore fails to induce the cells to absorb the therapeutic agent, whereas an electrical field that is too high damages the tissue and/or the device delivering the high current/voltage required to drive the electroporation process.

The electrical field present at the cells of the cochlea is derived from the current density at the cells using a version of Ohm's Law:

E=σJ

where:
E=the electrical field at the cells;
σ=the resistivity of fluid in the cochlea at the time of electroporation (e.g., the resistivity of the therapeutic agent solution introduced into the cochlea prior to electroporation; and
J=the current density at the cells

The current density in a cross-sectional area of the cochlea can be approximated by:

J=I/A

where:
I=the current delivered through the electroporation electrode contacts; and
A=the cross-sectional area through which current flows (e.g., cross-sectional area of the cochlea).

Combining these equations and rearranging the terms yields:

I=EA/σ

Therefore, the current delivered through the electroporation electrode contacts and the cross-sectional area of the cochlea are directly proportional.

The cross-sectional area of human, and most animal, cochleae varies widely along the cochlear length. Typically, the cross-sectional area of the cochlea is largest at the basal end and smallest at the apex. However, in conventional electroporation arrangements, the same electroporation signals (e.g., same current) are generated and delivered at every location along the length of the cochlea. Therefore, because of the varying cross-sectional area over the length of the cochlea, conventional electroporation arrangements produce varying electrical field strengths, over the length of the cochlea, that may be outside of the optimal range for electroporation (e.g., an electrical field that is too low at certain target nerve cells and/or an electrical field that is too high certain target nerve cells). For example, conventional electroporation devices can produce electrical field strengths at the basal end of the cochlea—where the cross-sectional area is large—that are too low to sufficiently electroporate target nerve cells. Conversely, conventional electroporation devices can produce electrical field strengths at the apical end of the cochlea—where the cross-sectional area is small—that are too large, meaning damage to the target cells or device can occur. For example, conventional electroporation devices can fail to electroporate the cochlea cells at the base of the cochlea for certain recipients and/or damage the tissue of the cochlea and/or the device itself at the apex of the cochlea for the same or other recipients.

Accordingly, presented herein are adaptive electroporation techniques in which the electroporation signals are adjusted/adapted based on the location within a body chamber, such as the cochlea, at which the electroporation electrical field is generated. For example, the electroporation signals (e.g., current signals) are delivered via one or more electrode contacts of an electroporation stimulation assembly (or “electroporation assembly”) inserted into the recipient's cochlea (or other body chamber, body cavity, other body area). One or more attributes/parameters of the electroporation signals are determined, selected, or otherwise set based on a location (e.g., insertion depth) of the one or more electrode contacts of an electroporation stimulation assembly within the cochlea. As a result, the electroporation electrical field generated by the electroporation signals is tailored to the position, and thus the cross-sectional area, of the cochlea location at which the one or more electrode contacts are located. This results in an electroporation electrical field at the target nerve cells that is within the optimal range for electroporation.

While the preceding description focuses on cochlea electroporation, it will be appreciated that the electroporation techniques described herein can apply to any suitable body chamber or area of the body. In certain examples, the body chamber can have a variable/changing cross-sectional dimension. The body chamber can be any chamber in human or animal body suitable for electroporation. The body chamber can be located in the inner ear, such as a cochlea or vestibular system, or can include neural tissue in a constrained space such as the spine.

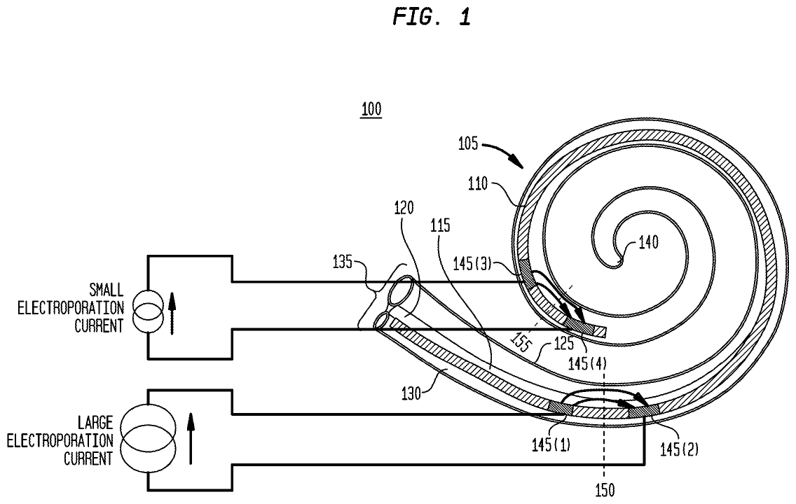

FIG. 1 is a schematic diagram 100 illustrating a cochlea 105 with an electroporation stimulation assembly 110 inserted therein, in accordance with certain embodiments presented herein. Cochlea 105 includes cochlear duct (also referred to as “scala media”) 115, basilar membrane 120, scala vestibuli 125, and scala tympani 130. Cochlea 105 includes a proximal/basal end (base) 135 and a distal/apical end (apex) 140. Cochlea 105 includes a variable cross-section that generally decreases over its end-to-end length. The cross-sectional area of cochlea 105 is greatest at base 135 of cochlea 105 and smallest at apex 140 of cochlea

Electroporation stimulation assembly 110 includes electroporation electrode contacts 145(1)-145(4). Electroporation electrode contacts 145(1) and 145(2) are positioned in proximity to basal location 150 (which is closer to base 135), and electroporation electrode contacts 145(3) and 145(4) are positioned in proximity to apical location 155 (which is closer to apex 140). Both basal location 150 and apical location 155 include target cells to be electroporated. While FIG. 1 depicts four electroporation electrode contacts (electroporation electrode contacts 145(1)-145(4)) positioned in proximity to basal location 150 and apical location 155, it will be appreciated that an electroporation stimulation assembly described herein can include any suitable number of electrode contacts positioned in proximity to any suitable location in a body chamber.

In operation, electroporation stimulation assembly 110 is positioned in/inserted into cochlea 105. Electroporation stimulation assembly 110 can be inserted manually or semi-automatically (e.g., by a human technician and/or any insertion tools), or automatically (e.g., robotically).

In one example, using electroporation electrode contacts 145(1) and 145(2), a first electroporation electrical field is generated at basal location 150 based on a cross-sectional area at basal location 150. The cross-sectional area at basal location 150 can be a cross-sectional area of cochlea 105 along the dashed line illustrating basal location 150. Furthermore, using electroporation electrode contacts 145(3) and 145(4), a second electroporation electrical field is generated at apical location 155 based on a cross-sectional area at apical location 155. The cross-sectional area at apical location 155 can be a cross-sectional area of cochlea 105 along the dashed line illustrating apical location 155.

In particular, electroporation electrode contacts 145(1)-145(4) can generate substantially constant electroporation electrical fields at basal location 150 and apical location 155, thus compensating for differences in cross-sectional area at basal location 150 and apical location 155. That is, the electroporation electrical fields at the target cells located at basal location 150 and apical location 155 can both have strength values that fall within a range that is suitable/optimal for electroporation. Thus, the target cells can be successfully electroporated regardless of their location in cochlea 105.

The electroporation electrical fields can be generated based on the cross-sectional areas of cochlea 105 using any suitable techniques. In one example, the actual cross-sectional areas of cochlea 105 can be explicitly determined through (calculated from) pre-operative imaging. The pre-operative imaging procedure can involve Computerized Tomography (CT), Magnetic Resonance Imaging (MRI), and/or other imaging methods. This can enable the electroporation electrical fields to be tailored to the individual recipient (cochlea 105) based on the anatomical knowledge (e.g., cochlear cross-section) acquired from the imaging. For example, the generated electroporation electrical fields be tailored for smaller-than-average or larger-than-average cross-sections of cochlea 105.

In another example, the cross-sectional areas can be estimated using values established for typical or average cochleae from prior data sets. The prior data sets can include a statistically significant number of cochlear cross-sectional areas that is sufficient to establish a typical cochlear cross-sectional area. The typical cross-sectional area can be used as a proxy for the cross-sectional areas of cochlea 105.

The insertion depth of a portion of the electroporation stimulation assembly 110 can serve as a proxy for the cross-sectional areas of cochlea 105. For example, the insertion depth can be correlated with the actual or estimated cross-sectional areas at a plurality of locations along the length of cochlea 105. The insertion depth can be determined based on visual markers on electroporation stimulation assembly 110 (e.g., manually), based on data captured during robotic insertion (e.g., automatically), and/or derived from measurements obtained from the electroporation stimulation assembly 110 (e.g., changes to electrical impedance as the electrode contacts are moved from a non-conductive medium, such as air, into a conductive medium, such as perilymph).

In yet another example, an angular insertion depth of a portion of electroporation stimulation assembly 110 can be determined. Like the insertion depth, the angular insertion depth can serve as a proxy for the cross-sectional areas of cochlea 105. In one example, the angular insertion depth can be determined from radiographic imaging.

In still another example, one or more electrical measurements can be obtained from cochlea 105 at basal location 150 and apical location 155 via electroporation stimulation assembly 110. The one or more electrical measurements can include one or more impedance measurements, such as a four-point impedance measurement. A four-point impedance measurement can involve passing a current between two outer electroporation electrode contacts and measuring an impedance induced between an inner pair of electroporation electrode contacts. Other impedance measurements can include a transimpedance measurement, a two-point impedance measurement, etc. Impedance is inversely proportional to the cross-sectional area of cochlea 105; therefore, the impedance measurement(s) can be used to infer, in-situ, the cross-sectional area at basal location 150 and apical location 155. For example, an intra-cochlear four-point impedance measurement taken (at least in part) by electroporation electrode contacts 145(1) and 145(2) can provide a value that is inversely proportional to the cross-sectional area of cochlea 105 at basal location 150, while an intra-cochlear four-point impedance measurement taken (at least in part) by electroporation electrode contacts 145(3) and 145(4) can provide a value that is inversely proportional to the cross-sectional area of cochlea 105 at apical location 155.

Other measurements that can be used to infer the cross-sectional area of cochlea 105 at a given depth of electroporation electrode contacts 145(1)-145(4) can include impedance spectroscopy measurements, pulsatile spectroscopy measurements, optical measurements, etc. Any suitable measurement can be employed.

Cochlear measurements corresponding to the cross-sectional area (e.g., imaging, insertion depth, angular insertion depth, electrical measurements, etc.) can be obtained immediately before the electroporation electrical fields are generated in cochlea 105. The measurements can be used to achieve an optimal electroporation field that is tailored to the anatomy of the cochlea (e.g. basal location 150 and apical location 155 in cochlea 105).

In addition to cross-sectional area, other factors can also influence the generated electroporation electrical fields in cochlea 105. One such factor is the resistivity of fluid in cochlea 105. Generally, the fluid in cochlea 105 at the time of electroporation is a combination of perilymph and the treatment material (e.g., gene therapy/DNA mixture). But there can be other components of the fluid composition that affect the overall resistivity. For example, the treatment material can be suspended in a carrier fluid such as sucrose or saline. Or contaminants, such as blood or bone dust, can be introduced during surgery. The size of electroporation stimulation assembly 110 itself can also impact the resistivity in cochlea 105 because its physical attributes determine the quantity of fluid displaced from the cochlea during insertion. The generated electroporation electrical field is generally proportional to the resistivity of the fluid.

Another factor that can also influence the generated electroporation electrical fields in cochlea 105 is the position of electroporation stimulation assembly 110 within cochlea 105. In particular, the relative proximity of electroporation electrode contacts 145(1)-145(4) to the cochlear wall (e.g., modiolus, lateral wall, mid-scala, etc.) or other structures within cochlea 105 can impact the generated electroporation electrical fields. For example, depending on the type of the electroporation sub-system, the narrowing cross-sectional area of cochlea 105 can decrease the relative proximity as electroporation stimulation assembly 110 advances apically. Closer proximity can result in greater electroporation electrical fields in the region surrounding electroporation electrode contacts 145(1)-145(4); farther proximity can result in reduced electroporation electrical fields in regions further from electroporation electrode contacts 145(1)-145(4).

The resistivity of the fluid and/or relative proximity of electroporation electrode contacts 145(1)-145(4) to the wall of cochlea 105 can be determined/measured using any suitable sensing technology discussed herein or otherwise known currently or developed hereafter. For example, impedance measurements can account for localised differences in resistivity as well as cross-sectional area. Four-point impedance, for example, is a function of the cross-sectional area and the resistivity of the fluid. Therefore, impedance measurements (or any other suitable measurements) can be used to generate an electroporation electrical field that accounts for the cross-sectional area of the body chamber and the resistivity of the fluid within the body chamber at the location of the electroporation electrode contacts (i.e., the localized cross-section and the localized resistance). The relative proximity of electroporation electrode contacts 145(1)-145(4) to the wall of cochlea 105 can also be similarly determined at basal location 150 or apical location 155, e.g., based on imaging or any other suitable techniques.

Based on the cross-sectional area, resistivity of the fluid and/or relative proximity of electroporation electrode contacts 145(1)-145(4) to the wall of cochlea 105, electroporation electrode contacts 145(1) and 145(2) can generate a first electroporation electrical field at basal location 150, and electroporation electrode contacts 145(3) and 145(4) can generate a second electroporation electrical field at apical location 155. In one example, the first and second electroporation electrical fields can be substantially constant electroporation electrical fields (e.g., within a suitable range for electroporation) at basal location 150 and apical location 155. In at least some examples, the current or voltage applied to a set of intra-cochlea electroporation electrode contacts is decreased as the electroporation electrode contacts are advanced apically within the cochlea. The current or voltage applied to the electroporation electrode contacts can be controlled responsive to the localized anatomy of the cochlea. For example, the current or voltage applied to the electroporation electrode contacts can be gradually decreased responsive to a progressive constriction in the basal region of the cochlea, and/or rapidly decreased responsive to an abrupt constriction in the apical region of the cochlea.

The first and second electroporation electrical fields can be generated based on a number of different measurements or data. For example, in certain embodiments, pre-operative imaging and/or prior data sets can be used in combination with a real-time measurement, such as insertion depth of electroporation stimulation assembly 110, angular insertion depth of electroporation stimulation assembly 110, one or more electrical measurements (e.g., impedance), spectroscopy measurements, optical measurements, etc.

Moreover, the first and second electroporation electrical fields can be generated using any suitable mechanism. In one example, the electroporation sub-assembly can apply a first current to electroporation electrode contacts 145(1) and 145(2) to produce the first electroporation electrical field at basal location 150, and the electroporation sub-assembly can apply a second current to electroporation electrode contacts 145(3) and 145(4) to produce the second electroporation electrical field at apical location 155. The first and second currents affect the induced voltages (e.g., electroporation electrical fields) in cochlea 105 so as to mitigate any adverse consequences of that voltage and/or ensure adequate electroporation of the target cells.

First and second current levels can be determined for use in generating the first and second electroporation electrical fields based on any suitable measurement or data. The electroporation sub-assembly can generate a first current at the first current level configured to produce the first electroporation electrical field at basal location 150 (corresponding to electroporation electrode contacts 145(1) and 145(2)), and the electroporation sub-assembly can generate a second current at the second current level configured to produce the second electroporation electrical field at apical location 155 (corresponding to electroporation electrode contacts 145(3) and 145(4)). In one specific example, the electroporation sub-assembly can take an impedance measurement at basal location 150 (corresponding to electroporation electrode contacts 145(1) and 145(2)) and, in response, generate the first current; and the electroporation sub-assembly can take an impedance measurement at apical location 155 (corresponding to electroporation electrode contacts 145(3) and 145(4)) and, in response, generate the second current.

In the example of FIG. 1, because the cross-sectional area at basal location 150 is greater than the cross-sectional area at apical location 155, the first current delivered through electroporation electrode contacts 145(1) and 145(2) is larger (e.g., proportionally larger) than the second current delivered through electroporation electrode contacts 145(3) and 145(4). That is, the electroporation current is adjusted in proportion to the cross-sectional area of cochlea 105 such that a substantially constant electroporation field is achieved at all locations within cochlea 105 proximate to the target cells. This can enable an optimal electroporation current to be delivered throughout cochlea 105. The current can be sufficiently high to cause efficient electroporation and sufficiently low to avoid inducing damaging voltages in cochlea 105. Thus, variable current electroporation can be provided based on various cross-sectional areas (e.g., diameters) of cochlea 105. As a result, different electroporation currents can be used for different locations (e.g., basal location 150 and apical location 155) in cochlea 105.

An Artificial Intelligence (AI) algorithm/process can be applied to an impedance measurement to determine a current level of a current configured to produce an electroporation electrical field. In one example, the AI algorithm can obtain an input in the form of one or more impedance or other measurements taken during electrode contact insertion and/or from any suitable intracochlear sensing method(s). Based on the input, the AI algorithm can provide an output in the form of a suggested current level of the current configured to produce the electroporation electrical field. The AI algorithm can be trained using data sets of measurements and corresponding current levels.

In addition to cross-sectional area, the resistivity of fluid in cochlea 105 and the proximity of electroporation electrode contacts 145(1)-145(4) to the cochlear wall or other structures within cochlea 105 can be at least partly responsible for determining the optimal or suitable electroporation current. The resistivity and/or proximity can enable delivery of electroporation pulses that are optimal for electroporation of the target cells. Using resistivity and/or proximity, the optimal electroporation current can be adjusted in real time, before applying the electroporation pulses.

The relationship between the geometry of the anatomical area (e.g., body chamber) at which the electroporation electrical field is generated, the relative position of the electrode(s) to anatomical structures (e.g., walls of the body chamber), the distance between the anode and cathode electrode contacts used to generate the electroporation electrical field, and the characteristics of the medium(s) between electrode contacts and cells to be electroporated is complex. Regardless of its exact nature, the relationship can be exploited to adjust the electroporation current based on, for example, the proximity and resistivity for optimal electroporation. In one example, a formula or expression combining one or more parameters (e.g., geometry, proximity, resistivity, distance, etc.) can be used to calculate the optimal electroporation current. In some embodiments, a measurement, such as a measured impedance, can account for various variables within the complex relationship and, such, can be used to more directly determine the optimal electroporation current for a given location (e.g., given cochlea location).

Thus, techniques are provided that enable electroporation parameters (e.g., current) to be controlled by anatomical or measured variation in the cochlea (in particular, cross-sectional area, but also resistivity of fluid and electrode contact proximity to, e.g., the modiolus). These techniques can enable intracochlear electroporation to employ a current that is effective but not damaging to the cochlear tissue or a cochlear implant. This can be an improvement over conventional electroporation, which uses the same current at multiple sites throughout the cochlear, resulting in either under-efficient electroporation in basal regions of the cochlear or unnecessarily high voltages in apical regions of the cochlear—both of which are undesirable. Using the techniques described herein, efficient electroporation electrical fields can be delivered without risking unnecessary damage to cochlear tissues or to a cochlear implant in-situ during the electroporation process. In particular, the applied electroporation signal (e.g., the electroporation currents and/or voltages) can be optimal for the part of the cochlea being electroporated. Thus, these techniques can avoid electroporation currents/voltages that are higher than are needed for effective electroporation, and/or electroporation currents/voltages that are too low and which will not provide effective electroporation. This can enable optimization of the applied electroporation signal for a particular region of the cochlear.

Certain electroporation parameters other than current can also be varied and this can influence the first and second electroporation electrical fields. For example, electrical parameters of the signal such as voltage level, number of pulses (e.g., number of pulses in a burst), pulse timing (e.g., timing of the applied pulses), pulse width, capacitor discharge, induction parameters (e.g., parameters for inducing a magnetic field externally to drive induction over an inductive link), etc. can be determined for use in generating the first and second electroporation electrical fields based on the cross-sectional area of cochlea 105 (for example, at basal location 150 and apical location 155). Like current, these electrical parameters can also/alternatively be adjusted based on intracochlear measurements and/or preoperative imaging measurements.

In one example, an electroporation voltage, rather than current, can be used to generate the first and second electroporation electrical fields in cochlea 105. The voltage used to drive a particular electroporation current is a function of the spreading impedance of the electroporation electrode contacts 145(1)-145(4). The spreading impedance refers to the impedance as the current exits or spreads out from electroporation electrode contacts 145(1)-145(4). The voltage is also a function of the voltage dropped as the current flows through cochlea 105. Thus, the voltage to achieve an optimal electroporation electrical field in cochlea 105 can take on a function of the form V=(a+b/A), where V is the required electroporation voltage, A is the cross-sectional area of the cochlea, and a and b are constants.

electrical field In one example, a moving electroporation electrical field can be generated by electroporation electrode contacts 145(1)-145(4), or a subset thereof, as they are moved through a spatial region proximate to the target cells (e.g., tissue) within the body. For instance, electroporation electrode contacts 145(1)-145(4) can be incrementally (e.g., robotically) inserted into and/or removed from cochlea 105. At each incremented location, a proxy measurement of the cross-sectional area (e.g., impedance) can be obtained. Based on the proxy measurement, the appropriate electroporation parameters can be chosen to generate an electroporation electrical field for the target cells (e.g., local to the incremented location). The parameters can be adjusted/changed/varied at each incremented location based on the cross-sectional area at that location.

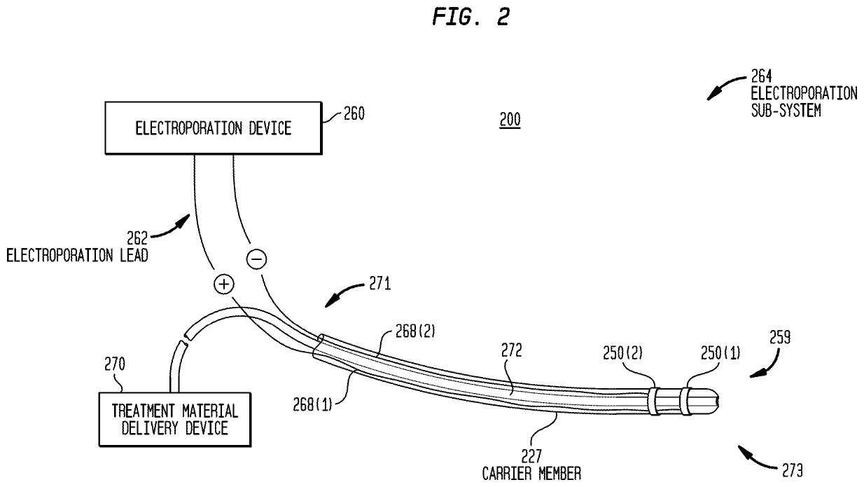

Certain embodiments presented herein can use dedicated electrode contacts that are used during electroporation only, and/or intra-chamber electrode contacts and one or more extra-chamber electrode contacts. The intra-chamber and extra-chamber electrode contact can be used post-operatively for stimulating the body chamber of a recipient. The dedicated electrode contacts can be integrated into the same carrier member (e.g., silicone or elastomer body) as the intra-chamber electrode contacts. Incorporating dedicated electrode contacts into the same carrier member as the intra-chamber electrode contacts can make the geometry (and hence the electrical field which governs the electroporation process) well-defined during electroporation. However, in other embodiments the dedicated electrode contacts can be physically separate from the carrier member in which the intra-chamber electrode contacts are disposed (e.g., part of an insertion tool, separate electrode contacts, etc.), referred to above as a “stand-alone electroporation sub-system.” FIG. 2 illustrate embodiments of one such stand-alone electroporation sub-system, in accordance with embodiments presented.

More specifically, FIG. 2 is a schematic diagram 200 of stand-alone electroporation sub-system 264, in accordance with certain embodiments presented herein. In one example, stand-alone electroporation sub-system 264 can be inserted, used to electroporate a body chamber, and then removed before an implant (e.g., a cochlear implant array, a vestibular implant, or a spinal implant) is inserted.

As shown, the stand-alone electroporation sub-system 264 comprises a carrier member (carrier member 227) in/on which two electroporation electrode contacts (electrodes) 250(1) and 250(2) are positioned/disposed. In the specific example of FIG. 2, the electroporation electrode contacts 250(1) and 250(2) are positioned at a distal end (tip) 259 of the carrier member 227. The electroporation electrode contacts 250(1) and 250(2) are electrically connected to an external electroporation device 260, which is configured to generate electroporation signals (e.g., current signals) to be delivered via the electroporation electrode contacts 250(1) and 250(2). More specifically, in this example, carrier member 227 includes two isolated electrical conductors (e.g. wires) 268(1) and 268(2), each of which terminate at one of the electroporation electrode contacts 250(1) and 250(2), respectively. The electrical conductors 268(1) and 268(2) extend through the electroporation lead 262 to the electroporation device 260.

As noted above, in order to perform electroporation, a treatment material is introduced into the proximity of the cells to be electroporated before (prior to) delivery of the electroporation electrical field. As such, in the embodiments of FIG. 2, the electroporation sub-system 264 comprises a treatment material delivery device 270. In these embodiments, a solution containing the treatment material to be transferred (e.g. transfected) into the chamber cells is pumped into the spatial region proximate to the target tissue (e.g., into the body chamber). More specifically, the treatment material delivery device 270 is fluidically coupled to a proximal end 271 of an elongate cannula 272 extending longitudinally through the carrier member 227. The cannula 272 has a distal opening 273 located at the distal end 259 of the carrier member 227 (e.g., for supplying treatment material to be transferred into cells). In some embodiments, the cannula 272 can have openings at other locations along the length of the carrier member 227 (e.g. between the electroporation electrode contact 250(1). 250(2)) either in addition or instead of the distal opening 273 shown in FIG. 2.

Treatment material delivery device 270 can be electrically operated (e.g., by a pump, pressurized reservoir, etc.) or can be manually operated (e.g., a syringe, manual pump, etc.) to supply a solution comprising the treatment material to be transferred into cells. The flow rate of the fluid delivery device can be set at a constant or variable flow rate to ensure an effective amount of the therapeutic agent is delivered to the appropriate location for electroporation.

As noted above, in accordance with the electroporation techniques presented herein, an electroporation electrical field is generated via (by) the electroporation electrode contacts 250(1) and 250(2) within the body chamber of a recipient. During electroporation, the electroporation signals can be delivered repeatedly, periodically, etc., via the electroporation electrode contacts 250(1) and 250(2).

In the embodiment of FIG. 2, two electroporation electrode contacts 250(1) and 250(2) are positioned at the distal end (tip) 259 of the carrier member 227. It is to be appreciated that the use of two electroporation electrode contacts is merely illustrative and that, in other embodiments, four, six, eight, ten, or more electrode contacts can positioned in/on the carrier member 227. In certain embodiments, the electroporation electrode contacts are arranged at, or begin at, proximate to the distal end 259 to enable electroporation of cells along the path of movement. However, in other embodiments, the electroporation electrode contacts can be arranged at other areas of the carrier member 227.

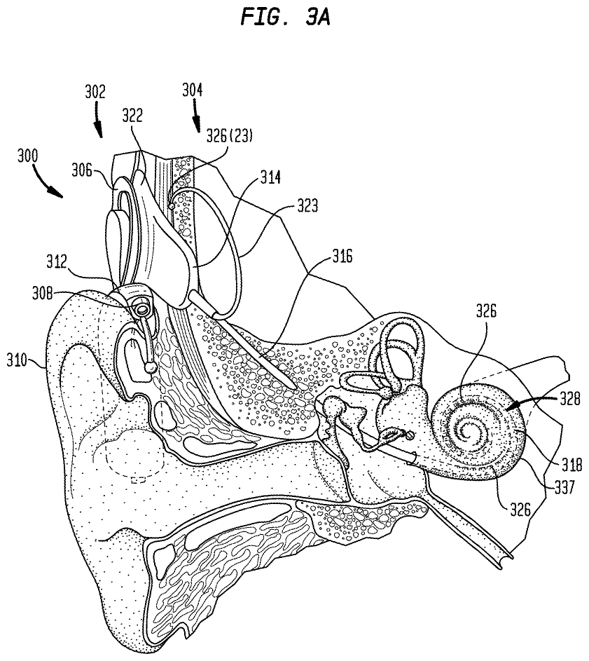

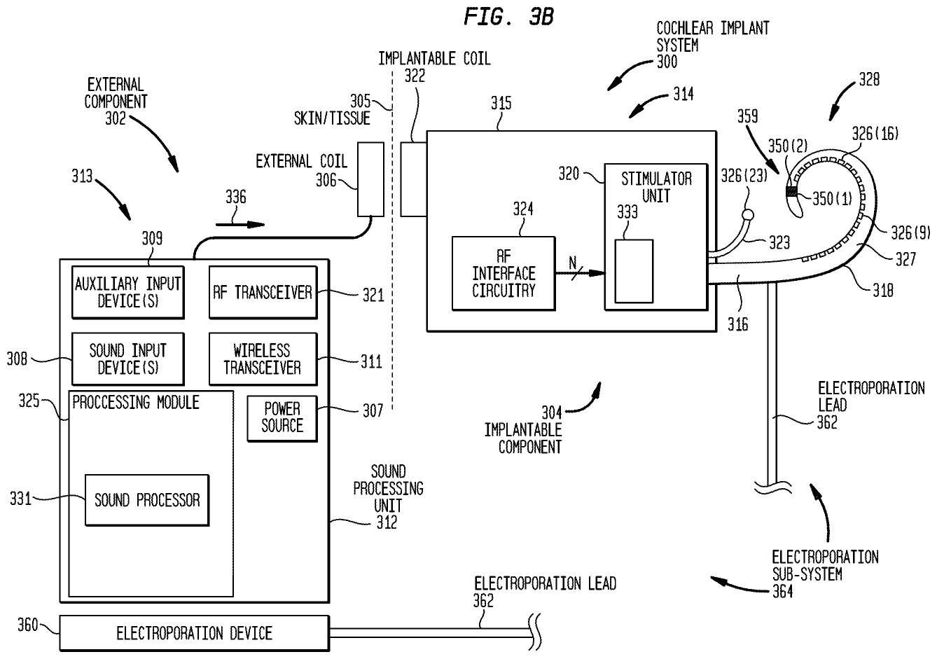

FIG. 3A is a schematic diagram of an exemplary cochlear implant system 300 configured to implement aspects of the techniques presented herein, namely configured for use in performing electroporation. FIG. 3B is a block diagram of the cochlear implant system 300. For ease of illustration, FIGS. 3A and 3B will be described together.

The cochlear implant system 300 comprises an external component 302 and an internal/implantable component 304, sometimes referred to herein as “cochlear implant 304.” The external component 302 is directly or indirectly attached to the body of the recipient and typically comprises an external coil 306 and, generally, a magnet (not shown in FIG. 3A) fixed relative to the external coil 306. The external component 302 also comprises one or more input elements/devices 313 for receiving input signals at a sound processing unit 312. In this example, the one or more one or more input devices 313 include sound input devices 308 (e.g., microphones positioned by auricle 310 of the recipient, telecoils, etc.) configured to capture/receive input signals, one or more auxiliary input devices 309 (e.g., audio ports, such as a Direct Audio Input (DAI), data ports, such as a Universal Serial Bus (USB) port, cable port, etc.), and a wireless transmitter/receiver (transceiver) 311, each located in, on, or near the sound processing unit 312.

The sound processing unit 312 also includes, for example, at least one battery 307, a radio-frequency (RF) transceiver 321, and a processing module 325. The processing module 325 can comprise a number of elements, including a sound processor 331.

In the examples of FIGS. 3A and 3B, the sound processing unit 312 is a behind-the-ear (BTE) sound processing unit configured to be attached to, and worn adjacent to, the recipient's car. However, it is to be appreciated that embodiments of the present invention can be implemented by sound processing units having other arrangements, such as an off-the-car (OTE) sound processing unit (i.e., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

Returning to the example embodiment of FIGS. 3A and 3B, the implantable component (cochlear implant) 304 comprises an implant body (main module) 314, a lead region 316, and an intra-cochlear stimulating assembly 318, all configured to be implanted under the skin/tissue (tissue) 305 of the recipient. The implant body 314 generally comprises a hermetically-sealed housing 315 in which RF interface circuitry 324 and a stimulator unit 320 are disposed. As described further below, the stimulator unit 320 comprises stimulation electronics 333. The stimulation electronics 333 comprises, among other elements, one or more current sources on an integrated circuit (IC). The implant body 314 also includes an internal/implantable coil 322 that is generally external to the housing 315, but which is connected to the RF interface circuitry 324 via a hermetic feedthrough (not shown in FIG. 3B).

As noted, stimulating assembly 318 is configured to be at least partially implanted in the recipient's cochlea 337. Stimulating assembly 318 includes a carrier member 327 and a plurality of longitudinally spaced intra-cochlear electrode contacts 326 disposed in/on the carrier member 327. The intra-cochlear electrode contacts 326 collectively form a contact or electrode array 328 configured to deliver electrical stimulation signals (current signals) to the recipient's cochlea and/or to sink stimulation signals from the recipient's cochlea. FIG. 3A illustrates a specific arrangement in which stimulating assembly 318 comprises twenty-two intra-cochlear electrode contacts 326, labeled as electrode contacts 326(1) through 326(22). It is to be appreciated that embodiments presented herein can be implemented in alternative arrangements having different numbers of intra-cochlear electrode contacts.

Stimulating assembly 318 extends through an opening in the recipient's cochlea (e.g., cochleostomy, the round window, etc.) and has a proximal end connected to stimulator unit 320 via lead region 316 and a hermetic feedthrough (not shown in FIG. 3B). Lead region 316 includes a plurality of conductors (wires) that electrically connect the electrode contacts 326 to the stimulator unit 320.

Also shown in FIG. 3A is an extra-cochlear electrode contact 326(23). The extra-cochlear electrode contact 326(23) is an electrode contact that is configured to, for example, deliver electrical stimulation to the recipient's cochlea and/or to sink current from the recipient's cochlea. The extra-cochlear electrode contact 326(23) is connected to a reference lead 323 that includes one or more conductors that electrically couple the extra-cochlear electrode contact 326(23) to the stimulator unit 320.

As described further below, the intra-cochlear electrode contacts 326(1)-326(22) and the extra-cochlear electrode contact 326(23) can be used post-operatively to stimulate the cochlea 337 of the recipient (i.e., operate as delivery or return paths for current signals to the cochlea 337) that evoke a hearing perception. As such, for ease of description, the intra-cochlear electrode contacts 326(1)-326(22) and the extra-cochlear electrode contact 326(23) are sometimes referred to herein as “stimulation electrode contacts.”

FIGS. 3A and 3B also illustrate that the stimulating assembly 318 includes two electrode contact contacts (electrodes) 350(3) and 350(3). As described further below, the electrode contacts 350(1) and 350(2) are electrically connected to an external electroporation device 360 (shown in FIG. 3B) via an electroporation lead 362 (e.g., one or more wires). As described elsewhere herein, the electrode contacts 350(1) and 350(2) can be used to electroporate the recipient's cochlea nerve cells during implantation of the stimulating assembly 318 into the cochlea 337. That is, the electrode contacts 350(1) and 350(2) can be configured to source, sink, or both source and sink electroporation signals (generated by external electroporation device 360) that result in the application of a electroporation electrical field to the nerve cells of the cochlea 337. Thereafter, the electrode contacts 350(1) and 350(2) can be electrically isolated from the external electroporation device 360 (e.g., electrically disconnected). Although shown as two electrode contacts located at the distal end 359 of the stimulating assembly 318, more than two electrode contacts can be present and used for electroporation.

Electrode contacts configured for use in performing electroporation, such as electrode contacts 350(1) and 350(2), are referred to as “electroporation electrode contacts.” The electroporation electrode contacts 350(1)/350(2), and the external electroporation device 360, are collectively referred to herein as a “electroporation sub-system” 364. In the embodiments of FIGS. 3A and 3B, the electroporation system 364 is referred to as being integrated with the cochlear implant system 300.

In the embodiments of FIGS. 3A and 3B, the electroporation electrode contacts 350(1) and 350(2) are structurally distinguishable from the intra-cochlear electrode contacts 326(1)-326(22) in that the electroporation electrode contacts are not connected to the stimulator unit 320 (whereas the intra-cochlear electrode contacts 326(1)-326(22) must be connected to the stimulator unit 320 to enable operation thereof). In particular, the high voltages associated with electroporation could damage the stimulator unit 320 and, as such, the electroporation electrode contacts 350(1) and 350(2) are electrically isolated from the stimulator unit 320. It also follows that, due to the electrical connection between the intra-cochlear electrode contacts 326(1)-326(22) and the stimulator unit 320, the intra-cochlear electrode contacts 326(1)-326(22) cannot be used to perform electroporation.

In accordance with certain embodiments, prior to electroporation, a therapeutic agent can first be delivered to the cochlea 337. Such a therapeutic agent can be delivered in a number of different manners, such as through an implantation tool, substance delivery device (e.g., lumen, syringe, etc.), a lumen within the carrier member 327, a coating on the carrier member 327, etc.

As noted, the cochlear implant system 300 includes the external coil 306 and the implantable coil 322. The coils 306 and 322 are typically wire antenna coils each comprised of multiple turns of electrically insulated single-strand or multi-strand platinum or gold wire. Generally, a magnet is fixed relative to each of the external coil 306 and the implantable coil 322. The magnets fixed relative to the external coil 306 and the implantable coil 322 facilitate the operational alignment of the external coil with the implantable coil. This operational alignment of the coils 306 and 322 enables the external component 302 to transmit data, as well as possibly power, to the implantable component 304 via a closely-coupled wireless link formed between the external coil 306 with the implantable coil 322. In certain examples, the closely-coupled wireless link is a radio frequency (RF) link. However, various other types of energy transfer, such as infrared (IR), electromagnetic, capacitive and inductive transfer, can be used to transfer the power and/or data from an external component to an implantable component and, as such, FIG. 3B illustrates only one example arrangement.

Sound processing unit 312 includes the processing module 325. The processing module 325 is configured to convert input audio signals into stimulation control signals 336 for use in stimulating a first ear of a recipient (i.e., the processing module 325 is configured to perform sound processing on input audio signals received at the sound processing unit 312). Stated differently, the sound processor 331 (e.g., one or more processing elements implementing firmware, software, etc.) is configured to convert the captured input audio signals into stimulation control signals 336 that represent stimulation signals for delivery to the recipient. The input audio signals that are processed and converted into stimulation control signals can be audio signals received via the sound input devices 308, signals received via the auxiliary input devices 309, and/or signals received via the wireless transceiver 311.

In the embodiment of FIG. 3B, the stimulation control signals 336 are provided to the RF transceiver 321, which transcutaneously transfers the stimulation control signals 336 (e.g., in an encoded manner) to the implantable component 304 via external coil 306 and implantable coil 322. That is, the stimulation control signals 336 are received at the RF interface circuitry 324 via implantable coil 322 and provided to the stimulator unit 320. The stimulator unit 320 is configured to utilize the stimulation control signals 336 to generate stimulation signals (e.g., current signals) for delivery to the recipient's cochlea via one or more stimulation electrode contacts 326(1)-326(22). In this way, cochlear implant system 300 electrically stimulates the recipient's auditory nerve cells, bypassing absent or defective hair cells that normally transduce acoustic vibrations into neural activity, in a manner that causes the recipient to perceive one or more components of the input audio signals.

More specifically, referring to the arrangement of FIGS. 3A and 3B, the electroporation electrode contacts 350(1) and 350(2) are electrically connected to the external electroporation device 360 via an electroporation lead 362. The electroporation electrode contacts 350(1) and 350(2) are used to electroporate the recipient's cochlea nerve cells. In certain examples, positioning the electroporation electrode contacts 350(1) and 350(2) at a distal end 359 of the carrier member, a lower voltage can be supplied to the electroporation electrode contact along the path, allowing a sufficient number of cells to be transferred.

FIGS. 3A and 3B illustrate an arrangement in which the cochlear implant system 300 includes an external component. It is to be appreciated that embodiments of the present invention can be implemented in cochlear implant systems having alternative arrangements and/or in other types of implantable medical devices.

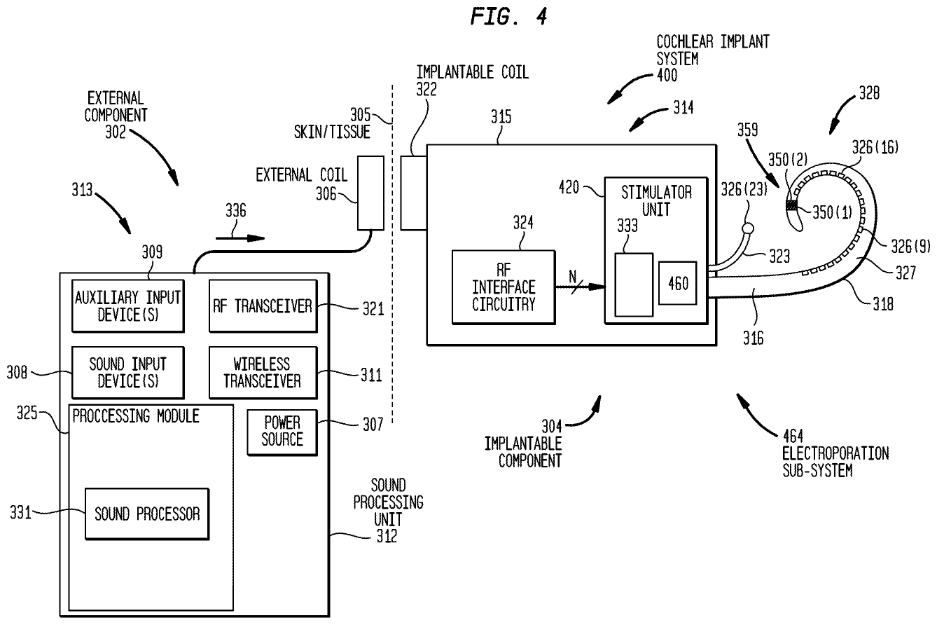

As noted, in the example of FIGS. 3A and 3B, the electroporation electrode contacts 350(1) and 350(2) are electrically connected to an external electroporation device 360. However, in certain alternative embodiments, the stimulator unit 320 can be designed to generate and handle the larger electroporation voltages. In such embodiments, the electroporation electrode contacts 350(1) and 350(2) could be connected to the stimulator unit 320, which in turn includes an electroporation signal generator, for generation and delivery of the electroporation signals (e.g., no connection to an external source is needed to generate the electroporation signals). Such an arrangement is shown in FIG. 4.

More specifically, FIG. 4 illustrates a cochlear implant system 400 that is substantially similar to cochlear implant system 300 of FIGS. 3A and 3B. However, in this example, the implantable component includes a stimulator unit 420 that can be designed to generate and handle the larger electroporation voltages. To this end, the stimulator unit 420 includes an on-board electroporation signal generator 460. The electroporation electrode contacts 350(1)/350(2), and the on-board electroporation signal generator 460, are collectively referred to herein as a “dynamic electroporation sub-system” 464. In the embodiment of FIG. 4, the dynamic electroporation system 464 is referred to as being integrated with the cochlear implant system 400.

The techniques described herein can provide a number of advantages to a cochlear implant system configured to provide an electroporation current (e.g., cochlear implant system 300 or cochlear implant system 400). Unlike conventional cochlear implant systems, which would deliver high electroporation currents and thereby induce high cochlear voltages-which can damage both the tissue of the cochlea and the implant itself-the cochlear implant systems described herein can avoid producing extraneous voltage or current beyond the amount involved in effectively inducing electroporation. As a result, the electroporation systems described herein can reduce tissue damage, more effectively deliver an electroporation stimulus, reduce the risk of damage to implantable electronics, simplify the circuitry required to generate an electroporation stimulus, and/or reduce the time involved in generating an electroporation stimulus.

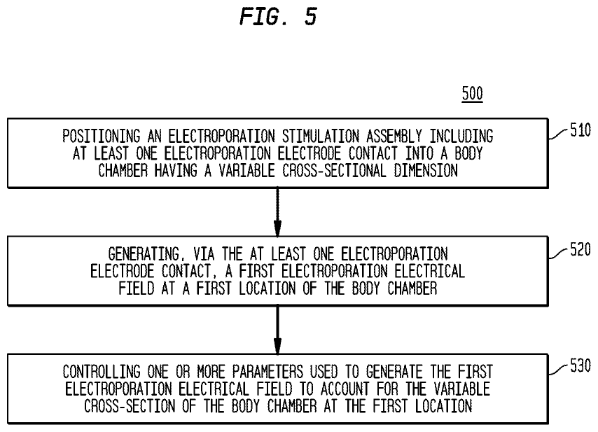

FIG. 5 is a flowchart of a method 500, in accordance with certain embodiments presented herein. At operation 510, an electroporation stimulation assembly including at least one electroporation electrode contact is positioned in a body chamber having a variable cross-sectional dimension. At operation 520, a first electroporation electrical field is generated at a first location of the body chamber using the at least one electroporation electrode contact. At operation 530, one or more parameters used to generate the first electroporation electrical field are controlled to account for the variable cross-section of the body chamber at the first location.

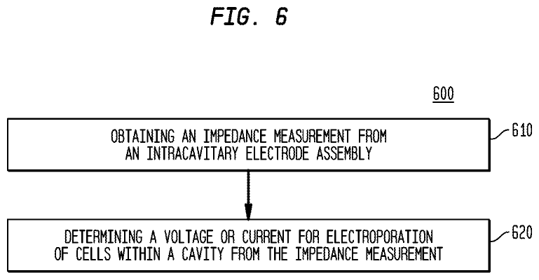

FIG. 6 is a flowchart of a method 600, in accordance with certain embodiments presented herein. At operation 610, an impedance measurement is obtained from an intracavitary electrode assembly. At operation 620, a voltage or current for electroporation of cells within a cavity is determined from the impedance measurement.

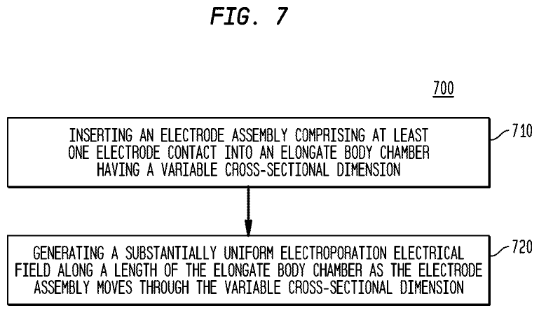

FIG. 7 is a flowchart of a method 700, in accordance with certain embodiments presented herein. At operation 710, an electrode assembly comprising at least one electrode contact is inserted into an elongate body chamber having a variable cross-sectional dimension. At 720, a substantially uniform electroporation electrical field is generated along a length of the elongate body chamber as the electrode assembly moves through the variable cross-sectional dimension.

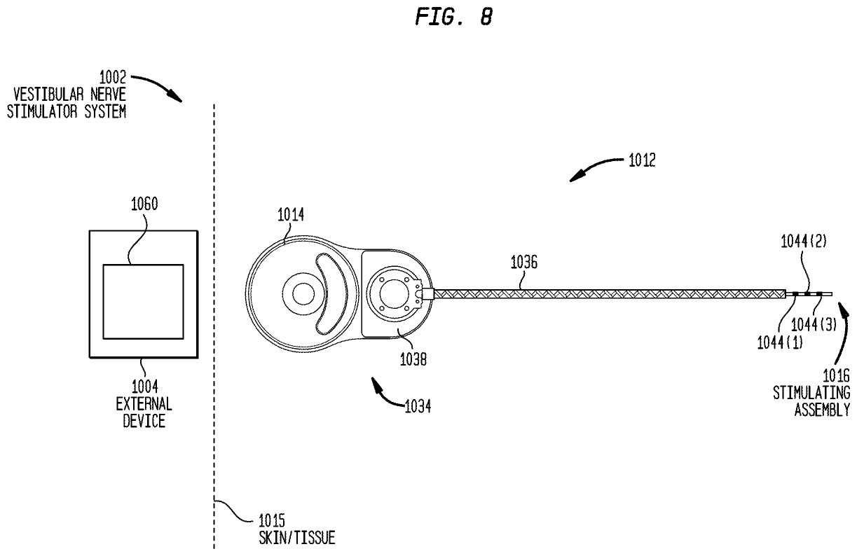

As previously described, the technology disclosed herein can be applied in any of a variety of circumstances and with a variety of different devices. For example, FIG. 8 illustrates an example vestibular stimulator with which aspects of the techniques presented herein can be implemented. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. These different systems and devices can benefit from the technology described herein.

As noted, FIG. 8 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

The stimulating assembly 1016 comprises a plurality of electrode contacts 1044(1)-(3) disposed in a carrier member (e.g., a flexible silicone body). In this specific example, the stimulating assembly 1016 comprises three (3) stimulation electrode contacts, referred to as stimulation electrode contacts 1044(1), 1044(2), and 1044(3). The stimulation electrode contacts 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrode contacts is merely illustrative and that the techniques presented herein can be used with stimulating assemblies having different numbers of stimulation electrode contacts, stimulating assemblies having different lengths, etc.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1-63. (canceled)

64. A method comprising:
obtaining an impedance measurement from an intracavitary electrode assembly; and
determining a voltage or current for electroporation of cells within a cavity from the impedance measurement.

65. The method of claim 64, wherein the method comprises:
obtaining a four-point impedance measurement from a body chamber via the intracavitary electrode assembly, and
applying the determined voltage or current to at least two electrodes of the intracavitary electrode assembly to create an electroporation electrical field within the body chamber.

66. The method of claim 64, wherein the method comprises:
positioning the intracavitary electrode assembly within an elongate body chamber;
obtaining at least one of a two-point impedance measurement, a four-point impedance measurement, or a transimpedance measurement within the elongate body chamber;
determining a voltage or current setpoint for electroporation of cells within the cavity from the at least one of the two-point impedance measurement, the four-point impedance measurement, or the transimpedance measurement within the elongate body chamber;
generating an electroporation electrical field within an elongate body chamber; and
using the voltage or current setpoint to control the voltage or current used to generate the electroporation electrical field.

67. The method of claim 64, wherein the method comprises:
positioning the intracavitary electrode assembly within an elongate body chamber; and
controlling the voltage or current supplied to the intracavitary electrode assembly at different locations within the elongate body chamber to produce a substantially uniform electroporation electrical field at the different locations.

68. The method of claim 64, further comprising:
applying an artificial intelligence process to the impedance measurement to determine a current level for electroporation of cells within the cavity.

69. The method of claim 64, wherein the method comprises inserting the intracavitary electrode assembly into the cavity, producing an electroporation electrical field within the cavity, and electroporating cells within or adjacent to the cavity.

70. The method of claim 64, wherein the method comprises inserting the intracavitary electrode assembly into an inner ear of a recipient and producing an electroporation electrical field at two or more distinct locations within the inner ear.

71. The method of claim 70, wherein inserting the intracavitary electrode assembly into the inner ear includes:
inserting the intracavitary electrode assembly in a vestibular system of the inner ear.

72. The method of claim 64 comprising:
inserting the intracavity electrode assembly into an elongate body chamber having a variable cross-sectional dimension; and
generating a substantially uniform electroporation electrical field along a length of the elongate body chamber as the intracavity electrode assembly moves through the variable cross-sectional dimension.

73. The method of claim 72, wherein generating the substantially uniform electroporation electrical field along a length of the elongate body chamber as the electrode assembly moves through the variable cross-sectional dimension, comprises:
dynamically adjusting a current used to produce the substantially uniform electroporation electrical field as the electrode assembly moves through the variable cross-sectional dimension.

74. The method of claim 73, further comprising:
applying a first voltage or current setpoint to generate the substantially uniform electroporation electrical field at a first location within the elongate body chamber; and
applying a second voltage or current setpoint to generate the substantially uniform electroporation electrical field at a second location within the elongate body chamber;
wherein the first voltage or current setpoint is greater than the second voltage or current setpoint.

75. The method of claim 73, further comprising:
moving the electrode assembly to a basal region of a recipient's cochlea;
applying a first voltage or first current to the at least one electrode contact at the basal region of the recipient's cochlea to generate the substantially uniform electroporation electrical field;
moving the electrode assembly to an apical region of the recipient's cochlea; and
applying a second voltage or second current to the at least one electrode contact at the apical region of the recipient's cochlea;
wherein the method comprises adjusting a voltage or current applied to at least one electrode contact to account for an insertion depth of the electrode contact within the recipient's cochlea and apply greater voltage or current at the basal region relative to the apical region.

76. The method of claim 64, further comprising:
determining an insertion depth of the electroporation stimulation assembly; and
controlling the one or more parameters used to generate the first electroporation electrical field responsive to the insertion depth of the electroporation stimulation assembly.

77. The method of claim 64, further comprising:
determining an angular insertion depth of a portion of the electroporation stimulation assembly; and
controlling the one or more parameters used to generate the first electroporation electrical field based on the angular insertion depth of the portion of the electroporation stimulation assembly.

78. The method of claim 64, further comprising:
obtaining a first impedance measurement at a first location within a body chamber, the first location having a first cross-sectional dimension;
determining a first current from the first impedance measurement that accounts for the first cross-sectional dimension, and applying the first current to the at least one electroporation electrode contact of the electroporation stimulation assembly to produce a first electroporation electrical field at the first location;
obtaining a second impedance measurement at a second location within the body chamber, the second location having a second cross-sectional dimension; and
determining a second current from the second impedance measurement that accounts for the second cross-sectional dimension, and applying the second current to the at least one electroporation electrode contact of the electroporation stimulation assembly to produce a second electroporation electrical field at the second location,
wherein the second electroporation electrical field is substantially the same as the first electroporation electrical field, and the second current is substantially different to the first current.

79. The method of claim 64, further comprising:
estimating a relative proximity of the intracavitary electrode assembly to a wall of a at the first location of the body chamber; and
controlling the voltage or current for electroporation of cells to account for the relative proximity of the intracavitary electrode assembly to the wall at the first location.

80. One or more non-transitory computer readable storage media comprising instructions that, when executed by a processor, cause the processor to:
receive intracavitary electrical measurements from an electrode array; and
determine one or more parameters for an electroporation electrical field from the intracavitary electrical measurements;
wherein the one or more parameters control a strength of the electroporation electrical field.

81. The one or more non-transitory computer readable storage media of claim 80, further comprising instructions that, when executed by a processor, cause the processor to:
estimate an insertion depth of the electrode array in an elongate body chamber of a recipient; and
control, using the insertion depth of the electrode array, the one or more parameters to compensate for anatomical variation of the elongate body chamber.

82. The one or more non-transitory computer readable storage media of claim 80, further comprising instructions that, when executed by a processor, cause the processor to:
receive a first intracavitary electrical measurement from an electrode array for a first location within a body chamber, and determine a first value for the one or more parameters for the electroporation electrical field at the first location within the body chamber from the first intracavitary electrical measurement; and
receive a second intracavitary electrical measurement from an electrode array for a second location within the body chamber, and determine a second value for the one or more parameters for the electroporation electrical field at the second location within the body chamber from the second intracavitary electrical measurement;
wherein the second value for the one or more parameters for the electroporation electrical field is substantially different to the first value for the one or more parameters for the electroporation electrical field, and the difference between the second value for the one or more parameters for the electroporation electrical field and the first value for the one or more parameters for the electroporation electrical field accounts for anatomical variations between the first location within the body chamber and the second location within the body chamber.

83. The one or more non-transitory computer readable storage media of claim 82, further comprising instructions that, when executed by a processor, cause the processor to:
produce a substantially same electroporation electrical field at the first location within the body chamber and the second location within the body chamber.

84. The one or more non-transitory computer readable storage media of claim 80, further comprising instructions that, when executed by a processor, cause the processor to:
obtain at least one of a two-point impedance measurement, a four-point impedance measurement, or a transimpedance measurement from the electrode array;
determine a voltage or current setpoint for the electroporation electrical field from the at least one of the two-point impedance measurement, the four-point impedance measurement, or the transimpedance measurement; and
use the voltage or current setpoint to control the voltage or current used to generate the electroporation electrical field.

85. The one or more non-transitory computer readable storage media of claim 80, further comprising instructions that, when executed by a processor, cause the processor to:
receive input that represents a depth of insertion of the electrode array within a cochlea of a recipient;
set a first voltage or current setpoint for an electroporation electrical field to be applied when the electrode array is in a basal region of the cochlea; and
set a second voltage or current setpoint for an electroporation electrical field to be applied when the electrode array is in an apical region of the cochlea;
wherein the second voltage or current setpoint is greater than the first voltage or current setpoint.
